SEQUENCE LISTING (5 sequences)

SEQ ID NO: 1 - DNA, 22 bp, Artificial Sequence
  PCR primer used to clone murine erythropoietin cDNA
gacagtgaccactttcttccag

SEQ ID NO: 2 - DNA, 24 bp, Artificial Sequence
  PCR primer used to clone murine erythropoietin cDNA
ggacagactggtaagaaggtaatg

SEQ ID NO: 3 - DNA, 18 bp, Artificial Sequence
  PCR primer used to clone murine erythropoietin cDNA
cagctaggcgcggagatg

SEQ ID NO: 4 - DNA, 19 bp, Artificial Sequence
  PCR primer used to clone murine erythropoietin cDNA
cagcagcatgtcacctgtc

SEQ ID NO: 5 - DNA, 243 bp, Artificial Sequence
  promoter sequence controlling expression of the EPO gene
gctagcgtcgtgcaggacgtgacatctagtgtcgtgcaggacgtgacatctagtgtcgtgcaggacgtgacatctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctg

(19) United States
(12) Patent Application Publication
(10) Pub. No.: US 2008/0125387 A1
(43) Pub. Date: May 29, 2008

(54) Anemia

(76) Inventors: Katie Mary Binley, Oxford (GB); Susan Mary Kingsman, Oxford (GB); Stuart Naylor, Oxford (GB)

Correspondence Address: FROMMER LAWRENCE & HAUG, 745 FIFTH AVENUE- 10TH FL., NEW YORK, NY, 10151, US

(21) Appl. No.: 11/705,645

(22) Filed: Feb. 12, 2007

Related U.S. Application Data
(63) Continuation of application No. 10/818,720, filed on Apr. 5, 2004, now abandoned.
Application No. 10/818,720 is a continuation-in-part of application No. 10/066,218, filed on Feb. 1, 2002, now abandoned.

(30) Foreign Application Priority Data
Jan. 31, 2002 (GB) 0202252.3

(51) Int. Cl. A61K 31/70; C12N 15/63; A61P 3/00

(52) U.S. Cl. 514/44; 435/320.1

(57) ABSTRACT

Disclosed is a viral vector containing a nucleic acid sequence encoding erythropoietin (Epo), in operable linkage with an HRE expression control sequence, as well as uses of the vector; for instance, in preparing a medicament. Also provided are methods for treating anemia, can involve administering the vector to a patient, wherein expression of Epo is physiologically regulated such that hematocrit levels of the patient are corrected and maintained.

### CROSS REFERENCE TO RELATED APPLICATIONS

This application is a continuation-in-part of U.S. application Ser. No. 10/066,218, filed on Feb. 1, 2002 and claiming priority from British application No. GB 0202252.3, filed on Jan. 31, 2002.

Reference is made to: U.S. Pat. No. 6,265,390 (Methods For Expressing Nucleic Acid Sequences Using Nucleic Acid Constructs Comprising Hypoxia Response Elements), filed on Feb. 22, 1999, to U.S. Pat. No. 5,942,434 (Nucleic Acid Constructs Comprising Hypoxia Response Elements), filed on Dec. 12, 1996, to International application No. PCT/GB95/00322 (Targeting Gene Therapy), filed on Feb. 15, 1995, and published as WO 95/21927 on Aug. 17, 1995, to GB application Serial No. 9402857, filed on Feb. 15, 1994, to U.S. application Ser. No. 09/787,562 (Polynucleotide Constructs and Their Uses Thereof), filed on Jul. 6, 2002, and to U.S. application Ser. No. 10/008,610 (Lentiviral-Mediated Growth Factor Gene Therapy for Neurodegenerative Diseases), filed on Nov. 8, 2001.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art. Furthermore, authors or inventors on documents incorporated by reference into this text are not to be considered to be “another” or “others” as to the present inventive entity and vice versa, especially where one or more authors or inventors on documents incorporated by reference into this text are an inventor or inventors named in the present inventive entity.

### FIELD OF THE INVENTION

The present invention relates to an improved vector system and the use of said vector in the treatment of chronic anemia. In particular, the present invention relates to the construction and use of a novel vector system that directs regulated erythropoietin (Epo) gene therapy in a manner that physiologically corrects the hematocrit levels in a patient in need of such treatment.

### BACKGROUND OF THE INVENTION

Tissue hypoxia is the key physiological signal for increasing erythropoiesis via a direct effect on the expression of the Epo gene (Maxwell et al. (1993) Kidney Int. 44: 1149-1462). Upon hypoxic exposure, the kidney, and to a lesser extent, the liver, increase Epo synthesis up to 1000-fold. Epo then circulates through the blood to the bone marrow where it promotes maturation of erythrocytes (Ebert et al. (1999) Blood 94: 1864-1877). Defining the mechanism of hypoxic induction of Epo production led to the identification of a potent regulatory sequence in the Epo enhancer that bound a transcription factor. The factor was identified as a heterodimer with independently regulated subunits termed hypoxia inducible factor-1 (HIF-1). HIF-1 is ubiquitously expressed and the consensus HIF-1 binding sequences exist in a number of genes in addition to Epo and are termed hypoxia responsive enhancers or elements (HRE) (Wenger et al. (1997) Biol. Chem. 378: 609-616). Defining the hypoxic regulation of Epo has led to advancement in the general understanding of the cellular response to hypoxia. In fact, various natural and synthetic HRE containing promoters have been used to direct heterologous gene expression in response to hypoxia, for example in tumour cells, muscle and macrophages (U.S. Pat. Nos. 6,265,390 and 5,942,434, Binley et al. (1999) Gene Ther. 6: 1721-1727, Griffiths et al. (2000) Gene Ther. 7: 255-262, Shibata et al. (2000) Gene Ther. 7: 493-498).

Chronic anemia occurs when there is a decrease in oxygen carrying capacity of the blood due to a shortage of red blood cells (RBC). One of the underlying causes of chronic anemia is a failure in the production of the protein hormone Epo that regulates the formation of RBCs. This results in a dramatic reduction in the number of circulating RBCs, measured by the hematocrit. This is particularly evident in end stage renal disease (ESRD), cancer and some chronic inflammatory diseases such as rheumatoid arthritis (Goodnough et al. (2000) Blood 96: 823-833, Bron et al. (2001) Semin. Oncol. 28: 1-6). The reduction in RBCs reduces the ability of the blood to oxygenate tissues causing tissue hypoxia. The pathophysiological responses correlate with the severity of the hypoxia and range from fatigue and hypertension through to cardiovascular disease and heart failure. Current treatment of this class of anemia includes the regular intravenous administration of recombinant human Epo (rhEpo) several times a week. However, on a cost and convenience basis this treatment regime may not be suitable for all indications particularly in severe chronic anemia that requires continuous and frequent treatment. Consequently, there has been considerable interest in developing a gene therapy strategy for the delivery of Epo whereby the single administration of the Epo gene would ensure the long-term delivery of Epo.

To this end, numerous methods for Epo gene therapy were investigated as a means to find alternatives to rhEpo protein therapy. These methods utilized a range of gene therapy delivery vehicles such as plasmid DNA, and viral vectors (U.S. Pat. No. 6,211,163, Osada et al. (1999) Kidney International 55: 1234-1240, Dalle et al. (1997) Hematol. Cell Ther. 39: 109-113, Bohl et al. (1998) Blood 92: 1512-1517, EP 1013288, Rudich et al. (May 2000) J. Surg. Res. 90: 102-108, Zhou et al. (May 1998) Gene Ther. 5: 665-670, Svennson et al. (October 1997) Hum. Gene Ther. 8: 1797-1806, Beall et al. (March 2000) Gene Ther. 7: 534-539, Payen et al. (March 2001) Exp. Hematol. 29: 295-300, Tripathy et al. (November 1994) PNAS 91: 11557-11561, Klinman et al. (March 1999) Hum. Gene Ther. 10: 659-665, Maione et al. (April 2000) Hum. Gene Ther. 11: 859-868, Descamps et al. (August 1994) Hum. Gene Ther. 5: 979-985, Maruyama et al. (March 2001) Gene Ther. 8: 461-468, Verma (1999) J. Gene Med. 1: 64-66, Kessler et al. (November 1996) PNAS 93: 14082-14087, Seppen et al. (August 2001) Blood 98: 594-596), or transfer of ex vivo modified Epo expressing cells (Bohl et al. (1997) Nat. Med. 3: 299-305, Osborne et al. (August 1995) PNAS 92: 8055-8058, Villeval et al. (August 1994) Blood 84: 928-933, Serguera et al. (1999) Hum. Gene Ther. 10: 375-383).

However, these methods failed to demonstrate any genuine therapeutic effect on chronic anemia. This is because the Epo gene has been delivered to either normal animals (Rudich, Beall, Serguera, and Bohl (1998), as above), or to inappropriate models such as beta-thalassemic mice (Villeval (1994), Payen (2001), as above, Bohl et al. (2000) Blood 95: 2793-2798, Dalle et al.(1999) Gene Ther. 6: 157-161), or to acutely anemic animals, for example where the kidneys have been severely damaged (Hamamori et al. (1995) J. Clin. Invest. 95: 1808-1813). As such, measurements of the hematocrit in these models are not a true indicator of therapy in that they are taken against baseline normal hematocrit levels or as a transient rise in the acute anemia environment. Furthermore, in many of these models, the introduction of the Epo gene results in a relentless rise in the hematocrit causing the opposite of anemia, polycythemia, a state characterized by having too many RBCs (Bohl et al. (2000), as above), which often requires frequent phlebotomy to reduce the risk of thrombosis (Rudich (2000), Zhou (1998), as above). It is believed that a consistently high hematocrit increases the risk of hypertension, heart failure and thrombosis. Thus, the state of the art represents that a method for providing meaningful Epo gene therapy in a clinical respect is both necessary and desirable.

In attempts to meet the need for regulating Epo gene therapy, researchers have developed systems that can be switched off by using a regulated promoter such as the Tetracycline or Rapamycin responsive promoters. However, to date, this approach has only been demonstrated to regulate the hematocrit above the normal baseline rather than to maintain normal levels (Ye et al. (1999) Science 283: 88-91, Bohl (1998), Rendahl (1998), and Bohl (1997), as above). In addition, the use of these extrinsic regulation systems in a clinical setting would require long-term maintenance and control of Epo gene expression, both of which would be costly and cumbersome, particularly since the addition of the pharmacological regulatory agents may interfere with other patient medications.

Setoguchi et al. (Blood, 94: 2946-2953, 1 Nov. 1994) utilize an adenoviral construct with human Epo gene (the gene itself including its 3′ 150 bp enhancer). The organization of the construct exploits the enhancer at the 3′ end of the human Epo gene in its natural position, the gene of which is under control of the adenoviral MLP promoter. The disadvantage with this approach is that it fails to produce physiologically-regulated expression of Epo.

Aebischer et al. (U.S. Pat. No. 5,952,226) utilize an encapsulated cellular implant to express the Epo gene. This technology is also described in Rinsch et al. (Human Gene Therapy, 8:1881-1889; Nov. 1, 1997). Rinsch et al. transformed isolated murine myoblasts in vitro with a vector expressing Epo. They then encapsulated the cells and implanted them into mice and rats kept under either normoxic or hypoxic conditions. The studies of Rinsch et al. and Aebischer et al. are distinct from the present invention. Rinsch et al. and Aebischer et al. created Epo-expressing cells ex vivo, and then transplanted the heterologous cells into an animal model. In contrast, the present Applicants have demonstrated that a vector system of the invention expressing Epo can be directly administered to animals and expressed in their own endogenous cells, such that hematocrit levels are corrected.

Accordingly, there remains a need in the art for a vector system suitable for the regulation of Epo which when functioning reproduces the physiological regulation of Epo, and thus allows patient hematocrit levels to be therapeutically corrected and maintained.

### SUMMARY OF THE INVENTION

The present invention provides an improved vector system suitable for the therapy of chronic anemia.

Thus in a first aspect, the present invention provides a vector system for the physiological regulation of Epo, the vector system comprising a nucleic acid sequence encoding erythropoietin (Epo) in operable linkage with an HRE expression control sequence, wherein the HRE expression control sequence includes two or more HRE expression control sequences, and the vector system, when administered to a host provides for the physiological regulation of Epo.

In a further aspect, the present invention provides the use of a vector system comprising a nucleic acid sequence encoding erythropoietin (Epo) in operable linkage with an HRE expression control sequence in the preparation of a medicament for the prophylaxis and/or treatment of anemia wherein the expression of Epo is physiologically regulated.

Organization of the construct of the present invention positions an HRE at the 5′ end of the construct in operable linkage with the promoter such that the HRE and promoter (creating a hypoxia inducible promoter/expression control sequence) controls expression of the Epo gene as set forth in FIG. 1A of this specification. In contrast to the present invention, the organization of the construct of Setoguchi et al. (Blood, 94: 2946-2953, 1 Nov. 1994) exploits the enhancer at the 3′ end of the human Epo gene in its natural position, the gene of which is under control of the adenoviral MLP promoter. Furthermore, the use of the construct as reported in Setoguchi et al., fails to segue to the surprisingly enhanced effects of the present invention reported herein, i.e., the near-perfect physiologically-regulated expression of Epo in the anemic environment of an art-recognized animal model.

Aebischer et al. (U.S. Pat. No. 5,952,226, and Human Gene Therapy, 8(16): 1840-1841, 1 Nov. 1997) utilize an encapsulated cellular implant to express the Epo gene. In contrast to the present invention, Aebischer et al. set forth an ex vivo approach rather than an in vivo approach, and furthermore, fail to teach or suggest the surprisingly enhanced effects of the present invention reported herein, i.e., the near-perfect physiologically-regulated expression of Epo in the anemic environment of an art-recognized animal model. Disadvantageously, the encapsulated cell technique of Aebischer et al. involves the surgical implant and explant of the capsule, whereas, in vivo administration of a gene therapy vector, as in the present invention, overcomes the need to surgically implant or explant the vehicle delivering the therapeutic gene.

In a further aspect still, the present invention provides the use of a vector comprising a nucleic acid sequence encoding erythropoietin (Epo) in operable linkage with an HRE expression control sequence in the preparation of a medicament for maintaining and correcting the hematocrit levels of a patient.

According to the above aspects of the invention, the HRE expression control sequence is advantageously associated with a promoter, preferably an HRE promoter within a vector system, to create a HRE promoter/expression control sequence. At least one HRE and/or HRE promoter/expression control sequence is in operable linkage with an Epo coding sequence. A vector system according to the invention directs the regulation of Epo expression in a surprising and unexpected manner and reproduces the near perfect physiologically-regulated expression of Epo in the anemic environment of an art recognized animal model.

Of course, the inventive vector is also useful for in vitro Epo expression, e.g., by contacting the vector with a suitable cell under conditions which allow for expression of the Epo, and optionally harvesting the expressed Epo, which can be used in the same fashion as other protein Epos.

Advantageously, the vector system can be any vector system, such as a viral vector system, e.g., retroviral, lentiviral, adenoviral, adeno-associated viral, and the like, or a non-viral vector system such as naked DNA, lipid complexed-DNA, or biolistic DNA delivery, DNA plasmid, and the like.

Advantageously, the vector system can be administered by any known route of delivery, such as intramuscular, intravascular, subcutaneous, or intraperitoneal administration. The skilled artisan, based on this disclosure and the knowledge in the art, including documents cited herein, can determine a route of administration, without any undue experimentation, including by considering such factors as the particular species of the patient and the particular vector.

Advantageously, the HRE can further be in operable linkage with any promoter, such as a viral promoter, or cellular promoter, that can be constitutive, inducible, or tissue-specific in function.

Advantageously, the Epo nucleic acid sequence can be synthetic or can be derived from any species of Epo, such as human Epo, non-human primate Epo, canine Epo, feline Epo, porcine Epo, bovine Epo, equine Epo, ovine Epo, and murine Epo.

Advantageously, the patient to be treated for chronic anemia may be any patient of a species such as human, non-human primate, canine, feline, porcine, bovine, equine, ovine, and murine. Advantageously, the present invention finds use in a clinical setting, which can include use in the veterinary field providing treatment to companion animals as well as farm animals. Advantageously, the Epo coding sequence and patient to be treated can be of the same species or of a different species.

The terms “comprises”, “comprising”, and the like are open, inclusive terms which do not exclude further elements; they can thus mean “includes”, “including” and the like.

These and other embodiments are disclosed or are obvious from and encompassed by, the following Detailed Description.

### BRIEF DESCRIPTION OF THE DRAWINGS

The following Detailed Description, given by way of example, but not intended to limit the invention to specific embodiments described, may be understood in conjunction with the accompanying Figures, incorporated herein by reference, in which:

FIG. 1A shows a diagrammatic representation of recombinant AAV-2 vectors used for this study. The AAV-CMVEpo and AAV-HREEpo virus vectors only differ in the nature of the promoter sequence. ITR indicates AAV-2 inverted terminal repeats; CMV, immediate/early promoter enhancer elements from CMV; HRE, hypoxia responsive promoter mEpo, murine erythropoietin; SV40 (pA); polyadenylation signal from the SV40 virus; Stuffer DNA, fragment of the 3′ β-galactosidase gene to ensure genome size is over 4 kb.

FIG. 1B shows proliferation of splenocytes incubated with supernatants from HT1080 cells transfected with pCMV/HRE-Epo plasmids. The assay shows the increased proliferation of the splenocyte cells when exposed to the supernatant from the pHRE-Epo transfected cells that have been exposed to hypoxia. The negative control consists of untreated cells, rhEpo indicates recombinant human Epo used as a positive control. Data are the mean relative light units per second values +/−SD of 3 samples.

FIG. 1C shows hypoxia regulated Epo expression is maintained in a rAAV vector. T47D cells were transduced with rAAV-2 vectors, AAV-CMVEpo and AAV-HREEpo. Supernatants were harvested 1 day (grey bars) and 4 days (white bars) post hypoxic treatment and analysed in an Epo ELISA assay. Data are the mean mIU/ml epo values +/−SD of 3 samples. The dotted line represents the detectable threshold of the assay.

FIG. 2 shows the skeletal muscle in the Epo-Tag^{h} transgenic mice (FIGS. 2B and 2D), which has increased vascularity compared to the parental wild type mice (FIGS. 2A and 2C). The skeletal muscle from Epo-TAg^{h} transgenic and parental wild type mice were sectioned transversely and immunologically stained for the endothelial cell marker, CD31 (FIGS. 2A and 2B) and the angiogenic factor, VEGF165 (FIGS. 2C and 2D).

FIG. 3A shows that AAV-HREEPO treated EPO-TAg^{h} transgenic mice display physiological correction of the hematocrit. Closed symbols represent EpoTAg^{h} groups and open symbols represent wild type groups; EpoTAg^{h} group (closed circles); wild-type-group (open circles); EpoTAg^{h} treated with AAV-CMVGFP (closed squares); wild-type treated with AAV-CMVEpo (open squares); EpoTAg^{h} treated AAV-CMVEpo (closed diamonds); wild-type treated with AAV-CMVEpo (open diamonds); EpoTAg^{h} treated with AAV-HREEpo (closed triangles); wild-type treated with AAV-HREEp (open triangles). Haematocrits are plotted as a mean value for 6 animals in each treatment group +/−SD.

FIG. 3B shows expansion of the hematocrit data from the mice treated with AAV-HREEpo; EpoTA^{h} mice (open squares); wild-type mice (open triangles). Hematocrit data from each individual animal treated with the AAV-HREEpo vector is plotted.

FIG. 4 shows analysis of the heart and spleens in the EpoTAg^{h} and wild-type mice before and after treatment with rAAVEpo vectors. (White bar) EpoTAg^{h} mice; (Black bar) wild-type mice; (Pale grey bar) EpoTAg^{h} mice treated with AAV-CMVEpo; (Dark grey bar) wild-type mice treated with AAV-CMVEpo. The average weight of organs is plotted +/− the standard deviation (n=3).

FIG. 5 shows histological analysis of the heart in untreated and rAAV-Epo treated Epo-TAg^{h} and wild-type mice. FIG. 5A shows an Epo-TAg^{h} heart with enlarged LV. FIG. 5B shows a wild type heart. FIG. 5C shows a Epo-Tag^{h} heart at day 70 post AAV-CMVEpo treatment. FIG. 5D shows a wild-type heart at day 70 post AAV-CMVEpo treatment.

FIGS. 6A-6D show electron micrographs of the hearts showing partial reversal of the cardiac hypertrophy.

FIGS. 7A and 7B show pONY8.4 series EIAV vectors comprising an HRE expression control sequence in operable linkage with a nucleotide sequence encoding Epo. These vectors are based on the EIAV pONY8 series of vector genomes. The vectors are self-inactivating (SIN), which eliminates the promoter activity of the viral 5′ LTR, thereby reducing the influence of the LTR promoter on the expression of the Epo gene. Transcription of the Epo coding sequence is driven from an internal HRE promoter, and preferably, the coding sequence is codon optimized to increase expression. The genome can also contain two other sequences that enhance expression, an upstream open-reading frame (ORF), which helps obviate the need for Rev in the system, and an expression enhancement sequence (EES) such as the WPRE.

### DETAILED DESCRIPTION OF THE INVENTION

Correcting anemia is a clinically important challenge as chronic anemia can lead to congestive heart failure that can be fatal if left untreated. The present invention achieves physiologically-regulated expression of the Epo gene and correction and maintenance of the hematocrit in a clinically relevant anemic environment. The present invention provides an optimized vector system comprising an HRE expression control sequence and optionally an HRE promoter in operable linkage with an Epo coding sequence, which vector system directs regulated Epo gene therapy in a surprising and unexpected manner by physiologically correcting and maintaining the hematocrit in a patient in need thereof.

The invention is also directed to the use of a vector system as herein described in the preparation of a medicament for the prophylaxis and/or treatment of chronic anemia, more specifically by mimicking the physiologically regulated expression of Epo.

The present invention is further directed to the use of a vector system as herein described in the preparation of a medicament for correcting and maintaining hematocrit levels in a patient. Hematocrit is the relative volume of blood occupied by erythrocytes.

With regard to the physiological correction and maintenance of the hematocrit level, it is meant for maintenance to encompass art-recognized treatment guidelines for chronic renal failure which seek to maintain hematocrit levels at 30-33% of the normal range of the hematocrit. (Kaufman et al. (1998) N Engl J Med 339: 578-583.) Normal levels of the hematocrit for males are 39-52%, and for females are 35-47%. (Anemia Work Group. (1997) NKF-DOQI clinical practice guidelines for the treatment of anemia of chronic renal failure. Am J Kidney Dis. 30(4 suppl 3): S192-S240.) Although, such guidelines recognized there was an increase in mortality if patients were maintained in the normal range, this was thought to be due to the effects of poor dosing control of Epo when provided by the current i.v./s.c. injection regimen. The guidelines also recognized that it can take some weeks for the effects of adjustments to appear making dose adjustment extremely difficult. (Asha et al. (1993) Am J Kid Dis. 22(2 suppl 1): 23-31.) Further, the rapid rise in hematocrit was thought to be causally related to the hypertension seen in a large proportion of patients treated. Raine et al. (1991) Am J Kidney Dis. 18(4 suppl 1): S76-S83, Besarab et al. (1998) N Engl J Med. 339: 584-590, Watson et al. (1990) Am J Med. 89: 432-435.) Thus, the present invention provides advantages over the aforementioned treatment paradigms, in that the method for Epo gene therapy of chronic anemia by administration of a vector system comprising an HRE expression control sequence in operable linkage with a gene encoding Epo, does not lead to rapid fluctuations, rather it provides a smoother restoration of the hematocrit described by the slow rise and smooth plateau of the hematocrit. This plateau of the hematocrit can be in the normal range of the hematocrit or it may be in the therapeutic range recognized by the aforementioned treatment guidelines. This slow rise and smooth plateau of the hematocrit is not possible with any other Epo therapies. In effect, the present invention offers a better clinical outcome than is possible with other Epo therapies known in the art.

It is therefore a feature of the instant invention to correct and maintain hematocrit levels in a patient. In the context of the invention, to “correct” means to raise the level of hematocrit to at least about 30-33% of normal levels, i.e., to about 12% to about 17% for males and to about 10% to about 16% in females, based on “normal levels” as stated above. Preferably, the level of hematocrit is corrected to about 40% of normal levels, more preferably to about 50% of normal levels, even more preferably to about 60% of normal levels. The level of hematocrit can be corrected to about 70%, 80% or 90% of normal levels. Advantageously, the level of hematocrit is corrected to 100% of normal levels, i.e., about 39% to about 52% in males and about 35% to about 47% in females. To “maintain” means that hematocrit levels remain within the range of corrected levels.

The present invention provides for the use of HREs, or hypoxically-inducible promoters/enhancers (expression control sequences), such as HREs derived from Epo, PGK-1 (EMBL database, accession no. M18735, at nucleotides 631 to 654 and 634 to 651), and LDH-A genes. The HREs of the invention may be chosen from those referred to herein, or they may be other HREs. It is expected that other hypoxically-inducible promoters or enhancers will be discovered as it has been shown that oxygen-sensing systems are widespread in mammalian cells and many genes are likely to be under hypoxic control (U.S. Pat. No. 6,265,390).

Advantageously, the nucleic acid construct according to the invention comprises at least one HRE, which confers hypoxic inducibility on the expression control sequence. There may be, for example, two or more HREs linked so as to increase hypoxic inducibility, and thus to increase the induction of the gene or genes under hypoxia. HREs may be chosen from among those referred to herein, or they may be other BREs. Oxygen-sensing systems are widespread in mammalian cells, and it is expected that other HREs having the fundamentally conserved structure and hypoxic inducible function, will be discovered (U.S. Pat. No. 6,265,390).

The construct according to the invention may comprise more than one, e.g., three or more copies of one of the Epo, PGK, LDH-A, or other HRE sequence given above. Additionally or alternatively, a longer portion of the Epo, PGK-1, LDH-A, or other enhancer or flanking sequence may be used in the construct, which longer portion comprises the HRE and part of the surrounding sequence (U.S. Pat. No. 6,265,390, as above). It is noted that regions of the Epo enhancer sequence have been well characterized (mouse Epo enhancer: EMBL accession no. X73471, Maxwell et al. (1993), U.S. Pat. No. 6,265,390, as above, and Semenza et al. (1992) PNAS 88: 5680-5684, and Blanchard et al. (1992) Mol. Cell. Biol. 12: 5373-5385).

The present invention provides for HREs that may be chosen so as to be operative in particular tissues or cell types to be targeted therapeutically, or they may be chosen to work in a wide range of tissues or cell types. Advantageously, the HRE of the present invention can be further in operable linkage with a promoter, such as a viral or cellular promoter. The HRE of the invention finds use with constitutive promoters such as the cytomegalovirus (CMV) promoter, SV40 early promoter, mouse mammary tumor virus LTR promoter, adenovirus major late promoter (MLP), and the Rous sarcoma virus (RSV) promoter, inducible promoters such as the murine metallothionein promoter, and tissue-specific promoters. Such promoter sequences are commercially available from, e.g. Stratagene (San Diego, Calif.). As to cytomegalovirus promoters, mention is made of U.S. Pat. Nos. 6,156,567 and 6,090,393, involving truncated CMV promoters, as well as U.S. Pat. Nos. 4,963,481 and 5,168,062.

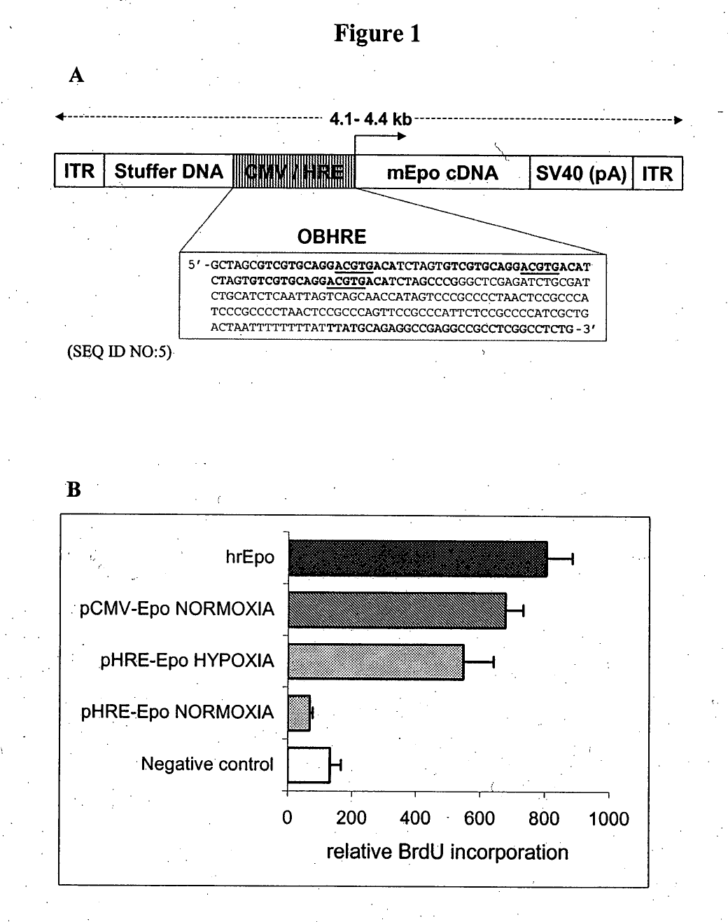

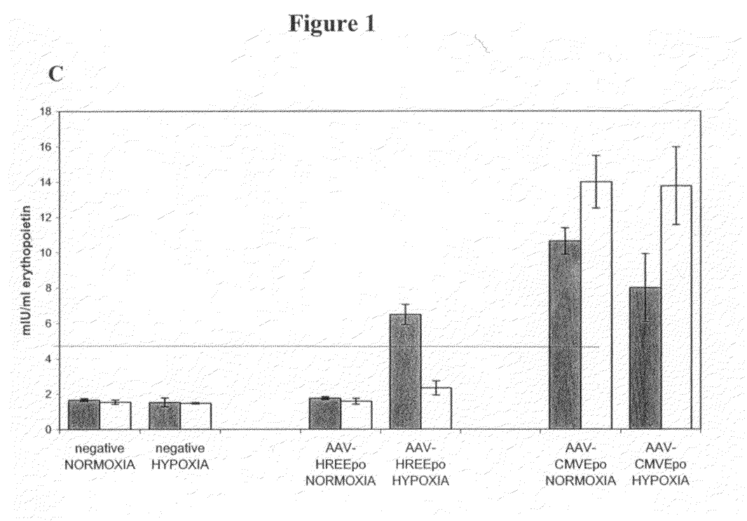

Organization of the construct of the present invention positions an HRE at the 5′ end of the construct optionally in operable linkage with the promoter such that the HRE and promoter (creating a hypoxia inducible promoter/expression control sequence) controls expression of the Epo gene as set forth in FIG. 1A of this specification.

The present invention provides a vector system which can be viral or non viral. Gene delivery of the Epo gene has been accomplished using a variety vectors such as retroviral, lentiviral, adenoviral, adeno-associated viral, naked DNA, lipid-complexed DNA, and biolistic DNA delivery (U.S. Pat. No.: 6,211,163, Osada et al. (1999), Dalle et al. (1997), Bohl et al. (1998), EP 1013288, Rudich et al. (May 2000, Zhou et al. (May 1998, Svennson et al. (October 1997), Beall et al. (March 2000), Payen et al. (March 2001, Tripathy et al. (November 1994), Klinman et al. (March 1999), Maione et al. (April 2000), Descamps et al. (August 1994, Maruyama et al. (March 2001), Verma (1999, Kessler et al. (November 1996), Seppen et al. (August 2001, (Bohl et al. (1997), Osborne et al. (August 1995), Villeval et al. (August 1994), Serguera et al. (1999), as above); see also U.S. Pat. Nos. 6,156,567, 6,090,393, 6,004,777, 5,990,091 and 6,130,066, and documents cited in these U.S. Patents, all incorporated herein by reference, for discussions of vectors that can be employed in the practice of the invention, including discussions of canine and human adenoviruses, and other vectors, e.g., poliovirus, herpesvirus, poxvirus, DNA vectors, etc. Adenoviruses useful in the practice of the invention can have deletions in the E1 and/or E3 and/or E4 regions, or can otherwise be maximized for receiving heterologous DNA; see, e.g., U.S. Pat. Nos. 6,156,567, 6,090,393, wherein an insertion of heterologous DNA can be in the E3 region or in the region located between the E4 region and the right ITR region. Mention is also made of U.S. Pat. Nos. 6,228,844, 6,214,804, 5,703,055, 5,693,622, 5,589,466, 5,580,859, 5,459,127, 5,264,618, which can involve vectors useful in the practice of the invention.

Preferably, the vector of the invention is a viral vector. The concept of using viral vectors for gene therapy is well known (Verma and Somia (1997) Nature 389:239-242). Even more preferably, the viral vector is a retroviral vector.

There are many retroviruses. For the present application, the term “retrovirus” includes: murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) and all other retroviridiae including lentiviruses. A detailed list of retroviruses may be found in Coffin et al (“Retroviruses” 1997 Cold Spring Harbour Laboratory Press Eds: J M Coffin, S M Hughes, H E Varmus pp 758-763).

Lentiviruses belong to the retrovirus family, and are notable because they can infect both dividing and non-dividing cells (Lewis et al (1992) EMBO J. 3053-3058). The lentivirus group can be split into “primate” and “non-primate”. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

Details on the genomic structure of some lentiviruses may be found in the art. By way of example, details on HIV and EIAV may be found from the NCBI Genbank database (i.e. Genome Accession Nos. AF033819 and AF033820 respectively). Examples of HIV-1 variants may be found in the HIV databases maintained by Los Alamos National Laboratory. Details of EIAV clones may be found at the NCBI database maintained by the National Institutes of Health.

Lentiviruses that are the subject of patents and patent publications and patent applications of Oxford Biomedica are advantageously employed in the practice of the invention.

During the process of infection, a retrovirus initially attaches to a specific cell surface receptor. On entry into the susceptible host cell, the retroviral RNA genome is then copied to DNA by the virally encoded reverse transcriptase, which is carried inside the parent virus. This DNA is transported to the host cell nucleus where it subsequently integrates into the host genome. At this stage, it is typically referred to as the provirus. The provirus is stable in the host chromosome during cell division and is transcribed like other cellular genes. The provirus encodes the proteins and other factors required to make more virus, which can leave the cell by a process sometimes called “budding”.

Each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′end of the RNA. The sizes of the three elements can vary considerably among different retroviruses.

For the viral genome, the site of transcription initiation is at the boundary between U3 and R in the left hand side LTR and the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins. Some retroviruses have any one or more of the following genes that code for proteins that are involved in the regulation of gene expression: tat, rev, tax and rex.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction leads ultimately to infection by fusion of the viral membrane with the cell membrane.

Retroviruses may also contain “additional” genes that encode proteins other than gag, pol and env. Examples of additional genes include, in HIV, one or more of vif, vpr, vpx, vpu, tat, rev and nef. EIAV has, for example, the additional genes S2 and dUTPase.

Proteins encoded by additional genes serve various functions, some of which may be duplicative of a function provided by a cellular protein. In EIAV, for example, tat acts as a transcriptional activator of the viral LTR. It binds to a stable, stem-loop RNA secondary structure referred to as TAR. Rev regulates and co-ordinates the expression of viral genes through rev-response elements (RRE). The mechanisms of action of these two proteins are thought to be broadly similar to the analogous mechanisms in the primate viruses. The function of S2 is unknown. In addition, an EIAV protein has been identified, Ttm, which is encoded by the first exon of tat, spliced to the env coding sequence at the start of the transmembrane protein.

Retroviral vector systems have been proposed as a delivery system for inter alia the transfer of a nucleotide of interest (NOI), such as Epo, to one or more sites of interest. The transfer can occur in vitro, ex vivo, in vivo, or combinations thereof. Retroviral vector systems have even been exploited to study various aspects of the retrovirus life cycle, including receptor usage, reverse transcription and RNA packaging (reviewed by Miller, 1992 Curr Top Microbiol Immunol 158:1-24).

A recombinant retroviral vector particle is capable of transducing a recipient cell with an NOI. Once within the cell, the RNA genome from the vector particle is reverse transcribed into DNA and integrated into the DNA of the recipient cell.

As used herein, the term “vector genome” refers to both to the RNA construct present in the retroviral vector particle and the integrated DNA construct. The term also embraces a separate or isolated DNA construct capable of encoding such an RNA genome. A retroviral or lentiviral genome should comprise at least one component part derivable from a retrovirus or a lentivirus. The term “derivable” is used in its normal sense as meaning a nucleotide sequence or a part thereof, which need not necessarily be obtained from a virus such as a lentivirus but instead could be derived therefrom. By way of example, the sequence may be prepared synthetically or by use of recombinant DNA techniques. Preferably the genome comprises a psi region (or an analogous component which is capable of causing encapsidation).

The viral vector genome is preferably “replication defective” by which we mean that the genome does not comprise sufficient genetic information alone to enable independent replication to produce infectious viral particles within the recipient cell. In a preferred embodiment, the genome lacks a functional env, gag or pol gene.

The viral vector genome may comprise some or all of the long terminal repeats (LTRs). Preferably the genome comprises at least part of the LTRs or an analogous sequence that is capable of mediating proviral integration, and transcription. The sequence may also comprise or act as an enhancer-promoter sequence.

It is known that the separate expression of the components required to produce a retroviral vector particle on separate DNA sequences cointroduced into the same cell will yield retroviral particles carrying defective retroviral genomes that carry therapeutic genes (e.g. Reviewed by Miller 1992). This cell is referred to as the producer cell.

There are two common procedures for generating producer cells. In one, the sequences encoding retroviral Gag, Pol and Env proteins are introduced into the cell and stably integrated into the cell genome; a stable cell line is produced which is referred to as the packaging cell line. The packaging cell line produces the proteins required for packaging retroviral RNA but it cannot bring about encapsidation due to the lack of a psi region. However, when a vector genome according to the first aspect of the invention (having a psi region) is introduced into the packaging cell line, the helper proteins can package the psi-positive recombinant vector RNA to produce the recombinant virus stock. This can be used to transduce the NOI into recipient cells. The recombinant virus whose genome lacks all genes required to make viral proteins can infect only once and cannot propagate. Hence, the NOI is introduced into the host cell genome without the generation of potentially harmful retrovirus. A summary of the available packaging lines is presented in “Retroviruses” (1997 Cold Spring Harbour Laboratory Press Eds: J M Coffin, S M Hughes, H E Varmus pp 449).

Another approach is to introduce the three different DNA sequences that are required to produce a retroviral vector particle i.e. the env coding sequences, the gag-pol coding sequence and the defective retroviral genome containing one or more NOIs into the cell at the same time by transient transfection and the procedure is referred to as transient triple transfection (Landau & Littman 1992; Pear et al 1993). The triple transfection procedure has been optimised (Soneoka et al 1995; Finer et al 1994). WO 94/29438 describes the production of producer cells in vitro using this multiple DNA transient transfection method.

The components of the viral system that are required to complement the vector genome may be present on one or more “producer plasmids” for transfecting into cells.

The term “viral vector system” is used generally to mean a kit of parts which can be used when combined with other necessary components for viral particle production to produce viral particles in host cells. For example, the retroviral vector genome may lack one or more of the genes needed for viral replication. This may be combined in a kit with a further complementary nucleotide sequence or sequences, for example on one or more producer plasmids. By cotransfection of the genome together with the producer plasmid(s), the necessary components should be provided for the production of infectious viral particles.

Alternatively, the complementary nucleotide sequence(s) may be stably present within a packaging cell line that is included in the kit.

Self-inactivating (SIN) retroviral vector systems have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus. However, any promoter(s) internal to the LTRs in such vectors will still be transcriptionally active. This strategy has been employed to eliminate effects of the enhancers and promoters in the viral LTRs on transcription from internally placed genes. Such effects include increased transcription or suppression of transcription. This strategy can also be used to eliminate downstream transcription from the 3′ LTR into genomic DNA. This is of particular concern in human gene therapy where it may be important to prevent the adventitious activation of an endogenous oncogene. Yu et al., (1986) PNAS 83: 3194-98; Marty et al., (1990) Biochimie 72: 885-7; Naviaux et al., (1996) J. Virol. 70: 5701-5; Iwakuma et al., (1999) Virol. 261: 120-32; Deglon et al., (2000) Human Gene Therapy 11: 179-90.

In the context of the viral vectors of the present invention, deletion of the U3 in the 3′LTR of the viral construct enhances Epo expression by increasing the level of induction of the HRE expression control sequence.

By using producer/packaging cell lines, it is possible to propagate and isolate quantities of retroviral vector particles (e.g. to prepare suitable titres of the retroviral vector particles) for subsequent transduction of, for example, a site of interest (such as adult brain or muscle tissue). Producer cell lines are usually better for large scale production or vector particles.

Transient transfection has numerous advantages over the packaging cell method. In this regard, transient transfection avoids the longer time required to generate stable vector-producing cell lines and is used if the vector genome or retroviral packaging components are toxic to cells. If the vector genome encodes toxic genes or genes that interfere with the replication of the host cell, such as inhibitors of the cell cycle or genes that induce apoptosis, it may be difficult to generate stable vector-producing cell lines, but transient transfection can be used to produce the vector before the cells die. Also, cell lines have been developed using transient infection that produce vector titre levels that are comparable to the levels obtained from stable vector-producing cell lines (Pear et al 1993, PNAS 90:8392-8396).

Producer cells/packaging cells can be of any suitable cell type. Producer cells are generally mammalian cells but can be, for example, insect cells.

As used herein, the term “producer cell” or “vector producing cell” refers to a cell that contains all the elements necessary for production of retroviral vector particles. Preferably, the producer cell is obtainable from a stable producer cell line. Preferably, the producer cell is obtainable from a derived stable producer cell line. Preferably, the producer cell is obtainable from a derived producer cell line.

As used herein, the term “derived producer cell line” is a transduced producer cell line which has been screened and selected for high expression of a marker gene. Such cell lines support high level expression from the retroviral genome. The term “derived producer cell line” is used interchangeably with the term “derived stable producer cell line” and the term “stable producer cell line. Preferably the derived producer cell line includes, but is not limited to, a retroviral and/or a lentiviral producer cell.

Preferably the derived producer cell line is an HIV or EIAV producer cell line, more preferably an EIAV producer cell line.

Preferably the envelope protein sequences, and nucleocapsid sequences are all stably integrated in the producer and/or packaging cell. However, one or more of these sequences could also exist in episomal form and gene expression could occur from the episome.

As used herein, the term “packaging cell” refers to a cell which contains those elements necessary for production of infectious recombinant virus which are lacking in the RNA genome. Typically, such packaging cells contain one or more producer plasmids which are capable of expressing viral structural proteins (such as codon optimised gag-pol and env) but they do not contain a packaging signal.

The term “packaging signal” which is referred to interchangeably as “packaging sequence” or “psi” is used in reference to the non-coding, cis-acting sequence required for encapsidation of retroviral RNA strands during viral particle formation. In HIV-1, this sequence has been mapped to loci extending from upstream of the major splice donor site (SD) to at least the gag start codon.

Packaging cell lines suitable for use with the above-described vector constructs may be readily prepared (see also WO 92/05266), and utilised to create producer cell lines for the production of retroviral vector particles. As already mentioned, a summary of the available packaging lines is presented in “Retroviruses” (as above).

Also as discussed above, simple packaging cell lines, comprising a provirus in which the packaging signal has been deleted, have been found to lead to the rapid production of undesirable replication competent viruses through recombination. In order to improve safety, second generation cell lines have been produced wherein the 3′LTR of the provirus is deleted. In such cells, two recombinations would be necessary to produce a wild type virus. A further improvement involves the introduction of the gag-pol genes and the env gene on separate constructs so-called third generation packaging cell lines. These constructs are introduced sequentially to prevent recombination during transfection. Preferably, the packaging cell lines are second generation packaging cell lines. Preferably, the packaging cell lines are third generation packaging cell lines.

In these split-construct, third generation cell lines, a further reduction in recombination may be achieved by changing the codons. This technique, based on the redundancy of the genetic code, aims to reduce homology between the separate constructs, for example between the regions of overlap in the gag-pol and env open reading frames.

The packaging cell lines are useful for providing the gene products necessary to encapsidate and provide a membrane protein for a high titre vector particle production. The packaging cell may be a cell cultured in vitro such as a tissue culture cell line. Suitable cell lines include but are not limited to mammalian cells such as murine fibroblast derived cell lines or human cell lines. Preferably the packaging cell line is a primate or human cell line, such as for example: HEK293, 293-T, TE671, HT1080.

Alternatively, the packaging cell may be a cell derived from the individual to be treated such as a monocyte, macrophage, blood cell or fibroblast. The cell may be isolated from an individual and the packaging and vector components administered ex vivo followed by re-administration of the autologous packaging cells.

It is highly desirable to use high-titre virus preparations in both experimental and practical applications. Techniques for increasing viral titre include using a psi plus packaging signal as discussed above and concentration of viral stocks. As used herein, the term “high titre” means an effective amount of a retroviral vector or particle which is capable of transducing a target site such as a cell.

As used herein, the term “effective amount” means an amount of a regulated retroviral or lentiviral vector or vector particle that is sufficient to induce expression of the NOIs at a target site.

A high-titre viral preparation for a producer/packaging cell is usually of the order of 10⁵ to 10⁷ retrovirus particles per ml. For transduction in tissues such as the brain, it is necessary to use very small volumes, so the viral preparation is concentrated by ultracentrifugation. The resulting preparation should have at least 10⁸ t.u./ml, preferably from 10⁸ to 10⁹ t.u./ml, more preferably at least 10⁹ t.u./ml. (The titer is expressed in transducing units per ml (t.u./ml) as titred on a standard D17 cell line). Other methods of concentration such as ultrafiltration or binding to and elution from a matrix may be used.

The presence of a sequence termed the central polypurine tract (cPPT) may improve the efficiency of gene delivery to non-dividing cells. This cis-acting element is located, for example, in the EIAV polymerase coding region element. Preferably the genome of the present invention comprises a cPPT sequence.

Preferably, the viral genome comprises a post-translational regulatory element. For example, the genome may comprise an element such as the woodchuck hepatitis virus posttranscriptional regulatory element (WPRE). Zufferey et al., (1999) J. Virol. 73: 2886; Barry et al., (2001) Human Gene Therapy 12: 1103.

In the context of the viral vectors of the present invention, inclusion of the WPRE in the viral construct enhances Epo expression by reducing baseline normoxia expression thus increasing the overall fold induction of the HRE expression control sequence.

In addition, or in the alternative, the viral genome may comprise a translational enhancer.

The NOIs may be operatively linked to one or more promoter/enhancer elements. Transcription of one or more NOIs may be under the control of viral LTRs or alternatively promoter-enhancer elements. Preferably the promoter is a strong viral promoter such as CMV, and RSV, or is a cellular constitutive promoter such as PGK, beta-actin or EF1alpha. The promoter may be regulated or tissue-specific. In a preferred embodiment, the promoter may be muscle specific.

In addition to the physiological control achieved using the HRE expression control sequence of the invention, it is quite possible to control protein expression at the translational level depending on the nature of the RNA transcript. In addition, it is known that it is possible to select, from random pools, RNA sequences of 20 to 40 nucleotides, that bind quite tightly to a specific ligand used in the selection process (See A D Ellington and J W Szostak “In vitro selection of RNA molecules that bind specific ligands” Nature 1990 346: 818-822; R R White, B A Sullenger and C R Rusconi “Developing aptamers into therapeutics” J. Clin Invest. 2000 106: 929-934). Interestingly, the major applications seen for such observations has been the use of the RNA molecules as antagonists for various interactions occurring in cells, such as the HIV tat-TAR RNA interaction that facilitates HIV infection (White et al op.cit.). One publication has suggested using this mechanism as a way to control gene expression by inserting an aptamer into a message sequence then adding a cell permeable ligand for which the aptamer has been selected (G Werstuck and M R Green “Controlling Gene expression in living cells through small molecule-RNA interactions” Science (1998) 282:296-298 and WO00/20040). However, the molecules proposed for use were either aminoglycoside antibiotics such as kanamycin and tobramycin or Hoechst dyes. Thus the system does not propose to use innocuous compounds for this purpose but rather compounds with known toxicities or that have no history of human use. This system thus is subject to issues described above. It also shows effects at concentration of drugs in the hundreds of micromolar to millimolar range. Typically this is the kind of concentration that is extremely difficult to reach in patient tissue or blood stream by oral administration of small molecule drugs.

However, it is possible to avoid these problems by selecting from a large library of sequences, with many more rounds of selection (20 to 40), aptamers that bind to innocuous well-characterized compounds with a record of human use. Ideally these are orally available, with known pharmacokinetics with a T½>12 h. These compounds are selected to be able to enter the tissue where it is desired to control expression. For example, for neural tissue the known ability to cross the blood brain barrier is important. The aptamer sequence is then inserted in the gene, the expression of which is to be controlled, and the safe permeable molecule used to turn off protein expression as desired. Examples of such small drug molecules include prescription drugs such as tetracycline or doxycycline, but also many over the counter (OTC) drugs (see such as aspirin or other mild analgesics), or compounds on the FDA list of “generally recognized as safe” (GRAS) compounds. Other examples are nicotine (normally used to quit smoking) and other nucleoside analogues, and various food additives including color dyes etc. If single aptamer sequences are responsive but only partially suppress expression, multiple copies can be inserted. The gene, the expression of which is to be controlled, can, in general, be delivered to animals and patients by any of the available viral or non-viral vector systems. (See “The development of Human Gene Therapy” T. Friedmann Ed., Cold Spring Harbor Laboratory Press, 1999). It can be used to control or further control expression of a therapeutic gene such as Epo, or an accessory gene such as a selectable marker or expression of a viral protein of a viral vector. In the case of a viral vector this can also be used to create replicating vectors, the replication of which is controllable by administration of an outside agent.

In the design of retroviral vector systems it is desirable to engineer particles with different target cell specificities to the native virus, to enable the delivery of genetic material to an expanded or altered range of cell types. One manner in which to achieve this is by engineering the virus envelope protein to alter its specificity. Another approach is to introduce a heterologous envelope protein into the vector particle to replace or add to the native envelope protein of the virus.

The term pseudotyping means incorporating in at least a part of, or substituting a part of, or replacing all of, an env gene of a viral genome with a heterologous env gene, for example an env gene from another virus. Pseudotyping is not a new phenomenon and examples may be found in WO 99/61639, WO-A-98/05759, WO-A-98/05754, WO-A-97/17457, WO-A-96/09400, WO-A-91/00047 and Mebatsion et al 1997 Cell 90, 841-847.

It has been demonstrated that a lentivirus minimal system can be constructed from HIV, SIV, FIV, and EIAV viruses. Such a system requires none of the additional genes vif, vpr, vpx, vpu, tat, rev and nef for either vector production or for transduction of dividing and non-dividing cells. It has also been demonstrated that an EIAV minimal vector system can be constructed which does not require S2 for either vector production or for transduction of dividing and non-dividing cells. The deletion of additional genes is highly advantageous. Firstly, it permits vectors to be produced without the genes associated with disease in lentiviral (e.g. HIV) infections. In particular, tat is associated with disease. Secondly, the deletion of additional genes permits the vector to package more heterologous DNA. Thirdly, genes whose function is unknown, such as S2, may be omitted, thus reducing the risk of causing undesired effects. Examples of minimal lentiviral vectors are disclosed in WO-A-99/32646 and in WO-A-98/17815. Examples of EIAV vector series, including derivations necessary for the pONY8 series, can be found in WO99/32646; WO99/61639; WO0236170; and WO03/064665.

Thus, a preferable delivery system is devoid of at least tat and S2 (if it is an EIAV vector system), and possibly also vif, vpr, vpx, vpu and nef. More preferably, the system is also devoid of rev. Rev was previously thought to be essential in some retroviral genomes for efficient virus production. For example, in the case of HIV, it was thought that rev and RRE sequence should be included. However, it has been found that the requirement for rev and RRE can be reduced or eliminated by codon optimisation or by replacement with other functional equivalent systems such as the HTLV Rex/RxRE and the CTE MPMV system. As expression of the codon optimised gag-pol is REV independent, RRE can be removed from the gag-pol expression cassette, thus removing any potential for recombination with any RRE or RRE like sequence contained on the vector genome.

Codon optimisation has previously been described in WO99/41397. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

Codon optimisation has a number of other advantages. By virtue of alterations in their sequences, the nucleotide sequences encoding the packaging components of the viral particles required for assembly of viral particles in the producer cells/packaging cells have RNA instability sequences (INS) eliminated from them. At the same time, the amino acid coding sequence for the packaging components is retained so that the viral components encoded by the sequences remain the same, or at least sufficiently similar that the function of the packaging components is not compromised. Codon optimisation also overcomes the Rev/RRE requirement for export, rendering optimised sequences Rev independent. Codon optimisation also reduces homologous recombination between different constructs within the vector system (for example between the regions of overlap in the gag-pol and env open reading frames). The overall effect of codon optimisation is therefore a notable increase in viral titre and improved safety.

In one embodiment only codons relating to INS are codon optimised. However, in a much more preferred and practical embodiment, the sequences are codon optimised in their entirety, with the exception of the sequence encompassing the frameshift site.

The gag-pol gene comprises two overlapping reading frames encoding gag and pol proteins respectively. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimised. Retaining this fragment will enable more efficient expression of the gag-pol proteins.

For EIAV, the beginning of the overlap has been taken to be nt 1262 (where nucleotide 1 is the A of the gag ATG). The end of the overlap is at 1461 bp. In order to ensure that the frameshift site and the gag-pol overlap are preserved, the wild type sequence has been retained from nt 1156 to 1465.

Derivations from optimal codon usage may be made, for example, in order to accommodate convenient restriction sites, and conservative amino acid changes may be introduced into the gag-pol proteins.

In a highly preferred embodiment, codon optimisation was based on highly expressed mammalian genes. The third and sometimes the second and third base may be changed.

Due to the degenerate nature of the Genetic Code, it will be appreciated that numerous gag-pol sequences can be achieved by a skilled worker. Also there are many retroviral variants described which can be used as a starting point for generating a codon optimised gag-pol sequence. Lentiviral genomes can be quite variable. For example there are many quasi-species of HIV-1 which are still functional. This is also the case for EIAV. These variants may be used to enhance particular parts of the transduction process.

The strategy for codon optimised gag-pol sequences can be used in relation to any retrovirus. This would apply to all lentiviruses, including EIAV, FIV, BIV, CAEV, VMV, SIV, HIV-1 and HIV-2. In addition this method could be used to increase expression of genes from HTLV-1, HTLV-2, HFV, HSRV and human endogenous retroviruses (HERV), MLV and other retroviruses.

Codon optimisation can render gag-pol expression Rev independent. In order to enable the use of anti-rev or RRE factors in the retroviral vector, however, it would be necessary to render the viral vector generation system totally Rev/RRE independent. Thus, the genome also needs to be modified. This is achieved by optimising vector genome components. Advantageously, these modifications also lead to the production of a safer system absent of all additional proteins both in the producer and in the transduced cell.

As described above, the packaging components for a retroviral vector include expression products of gag, pol and env genes. In addition, efficient packaging depends on a short sequence of 4 stem loops followed by a partial sequence from gag and env (the “packaging signal”). Thus, inclusion of a deleted gag sequence in the retroviral vector genome (in addition to the full gag sequence on the packaging construct) will optimise vector titre. To date efficient packaging has been reported to require from 255 to 360 nucleotides of gag in vectors that still retain env sequences, or about 40 nucleotides of gag in a particular combination of splice donor mutation, gag and env deletions. It has surprisingly been found that a deletion of all but the N-terminal 360 or so nucleotides in gag leads to an increase in vector titre. Thus, preferably, the retroviral vector genome includes a gag sequence that comprises one or more deletions, more preferably the gag sequence comprises about 360 nucleotides derivable from the N-terminus.

The present invention provides for administration of the vector system by any route of administration, such as intramuscular, subcutaneous, intravascular, or intraperitoneal (U.S. Pat. No. 6,211,163, as above, and Seppen (2001), as above).

The present invention provides the use of any Epo coding sequence. This sequence can be synthetic or can be derived from a species of Epo such as human Epo, non-human primate Epo, canine Epo, feline Epo, porcine Epo, bovine Epo, equine Epo, ovine Epo, and murine Epo. It is known that there is a high degree of sequence homology among Epo sequences in mammals. In fact, it has been reported that human Epo is 91% identical to monkey Epo, 85% to cat and dog Epos, and 80% to 82% to pig, sheep, mouse and rat Epos (Wen et al. (1993) Blood 82: 1507-1516). See also, WO99/5486; EP 1013288; U.S. Pat. Nos. 5,952,226; 5,621,080; 5,888,774; 4,954,437; 4,703,008; and 5,547,933; Descamps et al. (1994), as above; Seppen et al. (2001), as above; Shoemaker et al. (1986) Mol. Cell. Biol. 6: 849-858; Beall (2000), as above; Suliman et al. (1996) Gene 171: 275-280; and MacLeod et al. (1998) Am. J. Vet. Res. 59: 1144-1148. Accordingly, the invention is useful for delivery of Epo to humans, and non-human vertebrates, e.g., non-human mammals, such as canines, felines, non-human primates, porcines, bovines, equines, ovines, etc. Indeed, a problem recognized in the art is that human Epo is administered to animals, such as dogs, for treating anemia and/or other maladies, eventually leading to an immune response against the human Epo, such that there is a need for delivery of Epo to a particular species, e.g., species-specific delivery of Epo (such as delivery of canine Epo to dogs); and, the present invention may address this problem by providing to a host a vector that encodes an Epo specific to that host (such as providing to a dog a vector encoding canine Epo), or an Epo in a form that does not give rise to the problems encountered with administering human Epo to animals such as dogs. In such an instance, the vector can be tailored to the host too. For instance, if the intended host is a dog, the vector can be a canine adenovirus, with the coding therein for the Epo advantageously coding for canine Epo.

The present invention also provides modified, truncated, mutein, and active forms of Epo. See, e.g., U.S. Pat. Nos. 5,457,089; 5,166,322; 4,835,260; and 5,106,954. With respect to Epos, see also U.S. Pat. Nos. 5,955,422; 5,756,349; 5621,080; 5,618,698; 5,547,933; 4,703,008; 5,856,298; 5,661,125; 5,106,760; 4,703,008; 5,856,298; 5,661,125; 5,106,760; 4,558,006; 5,574,018; 5,354,934; 5,013,718; and 4,667,016.

The Epo sequence can be, for example, a synthetic RNA/DNA sequence, a codon optimised RNA/DNA sequence, a recombinant RNA/DNA sequence (i.e. prepared by use of recombinant DNA techniques), a cDNA sequence or a partial genomic DNA sequence, including combinations thereof. It need not be an entire coding region. In addition, the RNA/DNA sequence can be in a sense orientation or in an anti-sense orientation. Preferably, it is in a sense orientation. Preferably, the sequence is, comprises, or is transcribed from cDNA. The Epo sequence may encode all or part of the protein of interest (“POI”), or a mutant, homologue or variant thereof. For example, the Epo sequence may encode a fragment that is capable of functioning in vivo in an analogous manner to the wild-type protein.

The term “mutant” includes an Epo amino acid sequence that includes one or more amino acid variations from the wild-type sequence. For example, a mutant may comprise one or more amino acid additions, deletions or substitutions. A mutant may arise naturally, or may be created artificially (for example by site-directed mutagenesis).

Here, the term “homologue” means an entity having a certain homology with the Epo nucleic acid sequence, or which encodes a protein having a degree of homology with the Epo protein. Here, the term “homology” can be equated with “identity”.

In the present context, a homologous sequence is taken to include an amino acid sequence that may be at least 75, 85 or 90% identical, preferably at least 95 or 98% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence which may be at least 75, 85 or 90% identical, preferably at least 95 or 98% identical to the subject sequence. Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily-available sequence comparison programs. These commercially available computer programs can calculate % homology between two or more sequences.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalizing unduly the overall homology score. This is achieved by inserting “gaps” in the sequence alignment to try to maximize local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimized alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8).

Although the final % homology can be measured in terms of identity, the alignment process itself is typically not based on an all-or-nothing pair comparison. Instead, a scaled similarity score matrix is generally used that assigns scores to each pair-wise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another.

The present invention provides that the patient to be treated for chronic anemia or age-related anemia may be any patient of a species such as human, non-human primate, canine (e.g., dog, puppy, elder dog), feline (e.g., domestic or household cat, kitten, elder cat), porcine (e.g., pig, boar), bovine (e.g., cow), equine (e.g., horse), ovine (e.g., sheep, lamb), and murine. Advantageously, the present invention finds use in a clinical setting, which can include use in the veterinary field providing treatment to companion animals as well as farm and/or production and/or sport animals. Advantageously, the Epo coding sequence and patient to be treated can be of the same species or of different species. While the art recognizes a potential problem in the art based upon the importance of using autologous genes for Epo expression in animal strains with differing immunological responsiveness, the art also recognizes that species differences between host and gene can be tolerated (Kessler et al. (1996) PNAS 93: 14082-14087). To this end, the present invention provides for the physiological regulation of Epo expression in an anemic environment such that it is believed that tight control of Epo expression should overcome the need to limit the method to the use of an Epo gene from the same species in need of such treatment.

More specifically, the present invention has arisen from a desire to seek a model of human clinical potential. To this end, it was reasoned that the Epo-TAg mouse (Maxwell (1993), as above) should have tissue hypoxia as a consequence of the chronic anemic state and that this could be sufficient to activate gene expression from a hypoxia responsive promoter. In theory, once sufficient Epo was produced to restore the red blood cell (RBC) level to normal, the tissues should revert to normoxia and the HRE should cease to drive transcription. This would reduce Epo production and ensure that polycythemia, which condition may be fatal, does not develop.

Applicants have now tested this concept by using a recombinant adeno-associated viral (AAV) vector to express murine Epo under the control of a constitutive promoter (CMV) or a hypoxia regulated promoter (HRE). The method of gene delivery was chosen because the vascularity of skeletal muscle allows for the distribution of secreted proteins. In addition, as the hypoxia signalling pathway is functional in muscle, AAV gives a good gene transfer to muscle, and in a clinical setting, skeletal muscle is easily targeted by injection. The effect of intramuscular delivery of these vectors on the hematocrit and organ structure of normal and EpoTAg^{h} mice has been assessed over a long term study. The data indicates that Epo can be delivered upon physiological demand to reverse a chronic state of anemia.

The vectors can be administered in quantities based on the Examples herein, or in quantities that are based on the quantities of vector employed in documents cited herein or in other literature or patents, or in quantities for in vivo expression, which is commensurate with doses of protein Epo typically given to the particular patient (e.g., human or non-human). The dose for a particular patient can be determined by the skilled artisan, from this disclosure and the knowledge in the art, based on factors typically taken into consideration in the medical and veterinary arts, such as the particular species of the patient, age, sex, weight, condition and nature of host, as well as LD.sub.50 and other screening procedures which are known and do not require undue experimentation. Dosages of expressed product (protein Epo) can range from a few to a few hundred micrograms, e.g., 5 to 500 μg; for instance, when EPO is administered to a human patient (average mass about 70 kg) subcutaneously, it is given at a dose of about 40,000 units per week and if an inadequate response is seen, the dose can be increased to about 60,000 units, or lowered to about 20,000 units, on a weekly basis, depending on the response generated. The inventive recombinant vector can be administered in any suitable amount to achieve expression at these dosage levels. The viral recombinants of the invention can be administered in an amount of about 10^{3.5} pfu; thus, the inventive viral recombinant is preferably administered in at least this amount; more preferably about 10⁴ pfu to about 10⁶ pfu; however higher dosages such as about 10⁴ pfu to about 10¹⁰ pfu, e.g., about 10⁵ pfu to about 10⁹ pfu, for instance about 10⁶ pfu to about 10⁸ pfu can be employed. Suitable quantities of inventive plasmid or naked DNA in plasmid or naked DNA compositions can be 1 μg to 100 mg, preferably 0.1 to 10 mg, but lower levels such as 0.1 to 2 mg or preferably 1-10 μg may be employed. The dose can be adjusted or determined so that the patient's hematocrit levels are corrected and/or maintained.

Inventive vectors or formulations containing inventive vectors can be readministered, e.g., periodically and/or when hematocrit levels of the patient drop below corrected and/or maintained levels.

Inventive vectors may be formulated for administration based on the Examples herein, or based on formulations employed in documents cited herein or in other literature or patents, and can contain excipients, carriers, diluents and the like employed in vector formulations suitable for veterinary or medical (pharmaceutical) purposes, i.e., the formulations can contain veterinarily acceptable and/or pharmaceutically acceptable carrier(s), diluent(s), excipient(s) and the like, such as water or a buffered saline,. physiological saline, glucose or the like with or without a preservative. The vector compositions can also be lyophilized for resuspension or dissolving into solution, e.g., mixture with a carrier, diluent or excipient at or about the time of administration. The compositions can contain auxiliary substances, such as wetting or emulsifying agents, pH buffer agents, gelling or viscosity enhancing additives, preservatives, colors, and the like.

Accordingly, the invention comprehends a kit wherein the vector composition in lyophilized form is provided in a container, and a carrier, excipient or diluent is provided in a separate container, for admixture with the vector, to form a solution or suspension of the vector, for administration. The containers are optionally in the same packaging; and, the kit optionally can include instructions for admixture and/or administration. Thus, the invention further comprehends methods for preparing the vectors, as well as methods for preparing medicaments containing the vectors. The methods for preparing the vectors comprise operably linking the HRE(s) and the Epo coding sequences, optionally with a promoter such as a CMV promoter; and, the methods for preparing the medicaments or formulations comprise admixing the vector with the pharmaceutically and/or veterinarily acceptable carrier, diluent or excipient.

The inventive vector or formulation containing the inventive vector or the Epo expressed from the inventive vector can be administered alone, or in combination with other therapies for anemia or conditions underlying or causing the anemia; and thus, the invention comprehends combination therapy including the inventive vector or a formulation containing an inventive vector or an expression product from an inventive vector.

The invention will now be described by way of the following non-limiting Examples, given by way of illustration.

### EXAMPLES

### Materials & Methods

### Normal and Anemic Mice:

The generation of the anemic (EpoTAg^{h}) transgenic mice in which the SV40 large T antigen marker gene is integrated in the regulatory sequence of the endogenous mouse Epo gene is described elsewhere (Maxwell (1993, as above). The breeding colony of Epo-TAg^{h} and normal (C57B16/CBA) mice used in this study was maintained at CAMR, Porton Down, Wiltshire. The female EpoTAg^{h} homozygote mice were generated from F1 breeding pairs of heterozygote females and homozygote males. The genotype was determined by hematocrit; homozygote 17.5+/−1-4%, heterozygote 35.5+/−4.1% compared to the normal 52%.

### Cell Lines:

The T47D and HT1080 cell lines (ECACC, Wiltshire, UK) were used to assess hypoxic regulation of the Epo expression vectors since they have previously been shown to show good hypoxic induction in vitro 22. The cells were maintained in RPMI 1640 or Dulbecco's modified Eagle's medium respectively supplemented with 10% (v/v) fetal calf serum, 2 mM glutamine and 2 mM non-essential amino acids (Sigma-Aldrich, Dorset, UK).

### Transient Transfections:

Typically, cells seeded in a 24-well dish were brought to 70% confluence and transfected with 0.21 μg of plasmid using the Fugene-6 transfection reagent (Boehringer Mannheim, Indianapolis, USA).

Hypoxia In vitro:

24 hours post-transduction or transfection, cells were either incubated for a further 16 hours under normoxic conditions in a standard incubator (21% O₂, 5% CO₂, 74% N₂) or under hypoxic conditions (0.1% O₂, 5% CO₂, 95% N₂) using a multigas incubator purchased from Heto-Holten (Allerod, Denmark).

### In vitro Biological Assay for Erythropoietin:

The functionality and regulation of the cloned Epo cDNA was verified using a biological spleen cell proliferation assay based on a published method (Krystal (1983) Exp. Hematol. 11: 649-660). Briefly, 2 to 3 month old mice (C57BL/6J×C3H/HeB) F1 hybrid weighing 25-35 g were given two consecutive daily intraperitoneal injections of 60 mg/kg phenylhydrazine hydrochloride. Spleens were isolated three days after the second injection. Single cell suspensions from the spleen were prepared 3 days after the second injection and seeded into black-walled 96-well plates black plates (Canberra Packard, Ontario, Canada) at a density of 4×10⁵ cells per well. Supernatants were collected from HT1080 cells five days post-transfection with either pCMV-EPO or pHRE-EPO plasmids and 1 μl added to the splenocyte cell cultures. As a positive control recombinant human Epo (rhEpo) was used at 500 U/ml. The splenocyte cell cultures were incubated for 22 hrs and then assayed for proliferation using a chemiluminescent BrdU assay (Roche, Mannheim, Germany).

Detection of Erythropoietin In vitro:

Erythropoietin was detected in cell supernatants using the Quantikine IVD Epo Elisa kit, detectable threshold 2 mU/ml, (R & D systems, Abingdon, Oxon).

### Histological Analyses:

Standard haematoxylin and eosin staining was carried out in order to assess cell morphology. For immunohistological analysis the tissue sections were air dried and then fixed in absolute ethanol for 10 minutes. Endogenous peroxidase activity was blocked with 0.3% H₂O₂ in methanol for 10 minutes. To block non-specific binding sections were incubated in normal goat serum for 10 minutes followed by incubation with the primary antibody. Rabbit polyclonal VEGF (Santa-Cruz, Sc-507) was used at a dilution of 1/10. Goat anti rabbit horseradish peroxidase conjugated secondary antibody was used at a dilution of 1/50. Peroxidase substrate (DAB, Vector) was added for 10 minutes, washed and then counterstained using Gill's haematoxylin. Biotinylated mouse monoclonal CD 31 (BD Biosciences, 09332A) was used at a dilution of 1/100. Staining was detected using an alkaline phosphatase conjugated streptavidin secondary antibody at a dilution of 1/300. Slides were washed in distilled water for 5 minutes and then incubated in NBT/BCIP substrate (Roche). Levamisole was added to this solution to block endogenous alkaline phosphatase activity as per the manufacturer's instructions. Slides were counterstained in Gill's haematoxylin.

The percentage of CD31 positive cells in the tissue sections was calculated by random, equally processed digital images using the Aequitas Image Analysis Software (Digital Data Ltd., Cambridge, UK).

For electron microscopy the hearts were dissected in to 1 mm cubes and immersion fixed in 1% gluteraldehyde/2.5% paraformaldehyde. Samples were washed in PBS and post fixed in 1% OsO4 in 0.1M phosphate buffer for 40 minutes, washed in distilled water overnight at 4C, dehydrated in alcohols and embedded in Durcupan resin. Ultra thin cross-sections of the myocardium were stained with uranyl acetate, followed by 1% lead citrate (Reynold's stain), and examined under the Philips 401 transmission electron microscope.

### Example 1

### Construction of Recombinant AAV Vectors

The murine erythropoietin cDNA was cloned via nested PCR on murine kidney cDNA (Quickclone cDNA, Clontech, UK) using two pairs of nested PCR primers:

| | |
|---|---|
| Primer set 1: | |
| 5′-GACAGTGACCACTTTCTTCCAG-3′, | (SEQ ID NO: 1) |
| 5′GGACAGACTGGTAAGAAGGTAATG-3′. | (SEQ ID NO: 2) |
| Primer set 2: | |
| 5′-CAGCTAGGCGCGGAGATG-3′, | (SEQ ID NO: 3) |
| 5′-CAGCAGCATGTCACCTGTC-3′. | (SEQ ID NO: 4) |

The mEpo PCR product was cloned in to the pUC 18 plasmid (Panvera Corp, Wisconsin, USA) and was subsequently removed as an XbaI-EcoRI fragment and cloned into the pCI-Neo (Promega, Southampton, UK) NheI-EcoRI sites to create pCMV-Epo. The CMV/IE promoter in pCMV-Epo was replaced with the OBHRE promoter (Boast et al. (1999) Hum. Gene Ther. 10: 2197-2208) to create pHRE-Epo. An oligonucleotide was cloned into the BamHI and SpeI restriction sites in the multiple cloning site of the

pSL1180 plasmid (Amersham Pharmacia Biotech, Buckinghamshire, UK) to generate the following restriction sites: BamHI-NheI-MluI-XhoI-StuI-NruI-BclI-SpeI-BglII.

The AAV-CMVEpo vector genome was constructed by creating a145 bp oligonucleotide consisting of the wild-type AAV-2 inverted terminal repeat (ITR) (Genbank Accession number: NC_{—}001401) flanked by BamHI and NheI compatible ends. The ITR was cloned sequentially in both reverse and forward orientation into the BamHI-NheI and SpeI and BglII sites of the modified pSL1180 vector. The CMV-Epo BsaBI-BglII fragment from pCMVEpo was cloned into the StuI-BglII sites of the modified pSL1180 vector together with a 1.7 kb BclI-BglI stuffer fragment from the LacZ gene such that the complete internal cassette is 4.2 kb. The AAV-HREEpo vector genome was created by exchanging the CMV/IE NotI-Eco47III promoter fragment in AAV-CMVEpo for the OBHRE NotI-XmnI promoter fragment in pHRE-Epo (FIG. 1A).

The recombinant AAV-2 vectors were produced according to the published method (Zhang et al. (1999) Hum. Gene Ther. 10: 2527-2537). AAV particles were determined by dot blot quantification of genome copy and direct comparison to a recombinant AAV vector expressing CMV-GFP of known biological titer.

### Example 2

### Hypoxia Mediated Regulation of Functional Murine Epo Expression In vitro

It was observed that a synthetic HRE multimer referred to as OBHRE can combine a good induction ratio with high level of expression comparable to that achieved by strong constitutive promoters such as the CMV promoter but only when the oxygen concentration is low (Boast et al. (1999), as above). The OBHRE promoter was inserted into plasmid and AAV-2 vectors to produce pHRE and AAV-HRE respectively (FIG. 1A). Similar vectors containing the human CMV promoter are pCMV and AAV-CMV. A cDNA for murine Epo was inserted into these vectors and GFP expressing vectors were used as negative controls. Murine Epo rather than human Epo was used to ensure that immune responses would not compromise the efficacy of the gene therapy. It was first confirmed that the murine Epo gene functioned in vitro. The production of mEpo in the culture supernatant of HT1080 cells, transfected with pHRE-Epo or pCMV-Epo and maintained in normoxia or hypoxia, was determined using a spleen cell proliferation assay (FIG. 1B). Both plasmids directed the expression of functional mEpo, but in the case of the pHRE-Epo, the expression was eight fold higher from cells maintained in hypoxia as compared to the cells maintained in normoxia. Similarly, the recombinant AAV vectors were transduced into T47D cells, placed in normoxia or exposed to hypoxia for 16 hours and then returned to normoxia (FIG. 1C). The secretion of mEpo into the supernatant was assessed in an Epo ELISA 1 day and 4 days after hypoxic induction. AAV-CMV directed mEpo expression increased during the four days in both normoxia and hypoxia whereas AAV-HRE directed mEpo expression increased from basal levels up to a similar maximum level only in the hypoxia exposed cultures as measured at day 1. By day 4, however, levels of mEpo had returned to baseline. These data indicated that by two assays the mEpo gene was functional and that the expression could be activated by hypoxia and switched off in normoxia. This reversible expression was the profile that would be required for a gene therapy vector that could deliver Epo under anemic conditions, but which would be shut down once normal oxygenation was restored.

### Example 3

### Hypoxia Mediated Regulation of Functional Murine Epo Expression In vivo

### Hypoxic Status of Skeletal Muscle in Epo-TAg Mice:

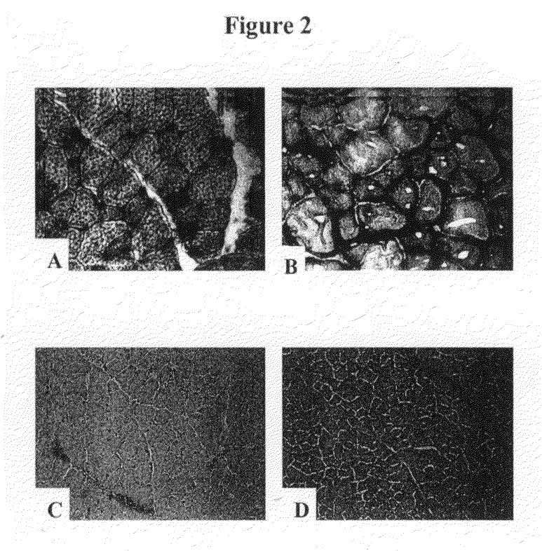

The concept of using a hypoxia responsive promoter to drive mEpo expression in skeletal muscle requires that there is tissue hypoxia. This was assessed prior to the study by examining the muscle for the expression of vascular endothelial growth factor (VEGF) and the consequent hypervascularisation. VEGF gene expression is activated by hypoxia, predominantly via the HIF-1 mediated transcriptional pathway, and stimulates endothelial cell proliferation and neovascularisation. This presumably is an attempt to compensate for the low oxygen tension in the tissue by increasing the blood flow/oxygen supply to the anemic limb. Hind limb skeletal muscle from the EpoTAg^{h} mice showed increased staining for VEGF and for CD31, an endothelial cell specific marker from 10.7%+/−5.1 in the EpoTAg^{h} compared to 7.4%+/−4.0 in the normal skeletal muscle (FIG. 2). These data indicated that the skeletal muscle was overexpressing VEGF and was therefore likely to be sufficiently hypoxic to activate the HRE, particularly in the young mice at the start of the study.

Regulated Delivery of Epo In vivo:

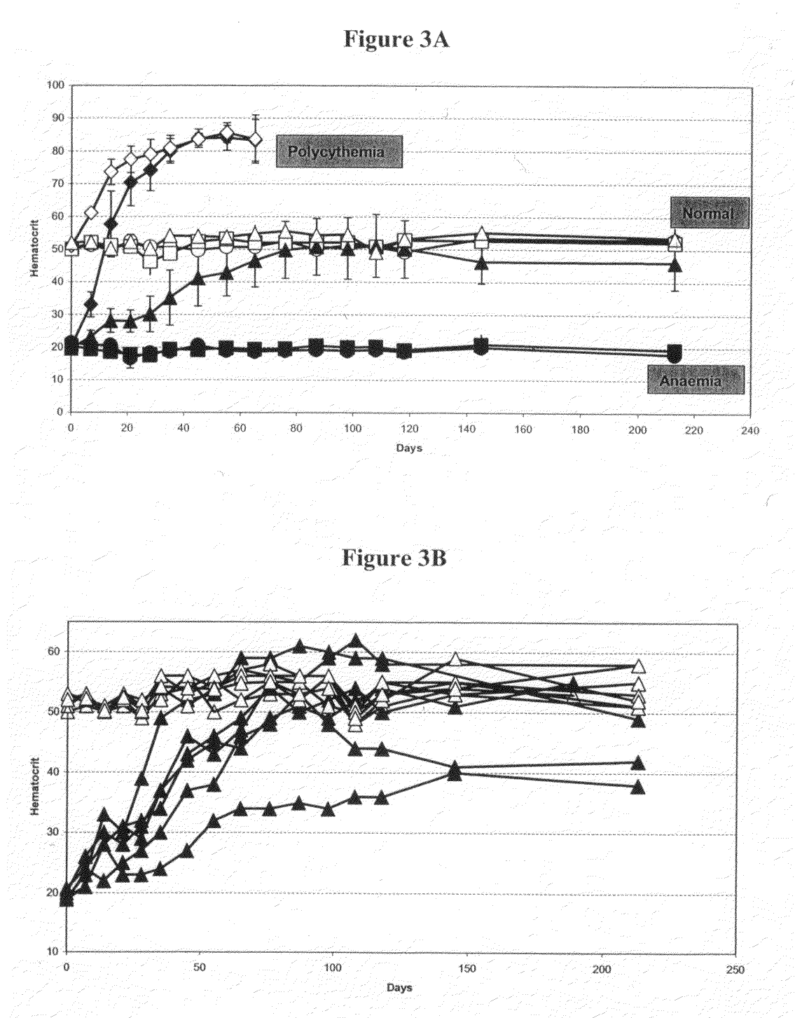

Twelve week old female mice were injected with a total dose of 1×10¹⁰ particles of recombinant AAV vector at four sites in the left hind-limb. Two 30 μl injections were made in to the quadriceps and two 20 μl in to the anterior tibialis muscles. Hematocrit measurements were made regularly over a period of 7 months (FIG. 3). The control vector was AAV-CMVGFP and this produced no change in the hematocrit in normal mice, which was maintained at about 52% (FIG. 3A, open squares) or in EpoTAg^{h} mice, which was maintained at about 18% (FIG. 3A, closed squares) throughout the duration of the study. These levels were identical to the untreated controls (FIG. 3A, normal mice, open circles; EpoTAg^{h} mice, closed circles). In marked contrast, when the normal and EpoTAg^{h} mice were injected with the constitutive Epo vector, AAV-CMVEpo, there was a dramatic rise in the hematocrit in both groups that was significant at 5 days and that increased to 85% after 35 days (FIG. 3A, diamond symbols). Two mice in this group died suddenly at day 60, by which time the blood in the remaining animals became too viscous to obtain samples for hematocrit analysis so the animals in these groups were sacrificed. However, a dramatically different result was obtained when the hypoxia regulated vector, AAV-HREEpo, was used. In normal mice (FIG. 3, open triangles) there was no effect on the hematocrit, it was virtually indistinguishable from the untreated and AAV-CMVGFP treated controls giving a peak hematocrit of 55.6%+/−1.8 at day 78. In the EpoTAg^{h} mice, the hematocrit began to rise steadily until at 75 days a plateau was reached. This plateau was at an average hematocrit of 54%, i.e., in the normal range (FIG. 3, closed triangles). This normal hematocrit was maintained up to 160 days when the study terminated. The response was remarkably consistent across all the treated animals and the individual data are shown in FIG. 3B. The hematocrits of the AAV-HREEpo treated normal mice are virtually super imposable. The hematocrits of the AAV-HREEpo treated EpoTAg^{h} mice showed some variation in terms of the rate of increase and plateau level. However, in no case did the hematocrit reach the levels obtained by the constitutive vector, and in all cases, plateau levels were within the normal range. The constitutive AAV-CMVEpo vector was highly toxic causing death or severe morbidity by 65 days. Whereas treatment with the hypoxia regulated AAV-HREEpo vector not only restored normal hematocrit, but also lead to the maintenance of these normal levels for the duration of the 7 month study.

### Organ Analysis of Animals:

It was desirable to determine if Epo gene therapy caused any structural changes to internal organs. Changes in red blood cell composition affect both the volume and pressure of the blood. In chronic anemia, this hemodynamic alteration leads to gradual development of cardiac enlargement (hypertrophy) as the cardiac output increases to compensate for the decreased oxygen carrying capacity of the blood. A significant increase in the hematocrit, a condition known as polycythemia greatly increases the viscosity of the blood leading to greater risk of thrombosis and heart failure.

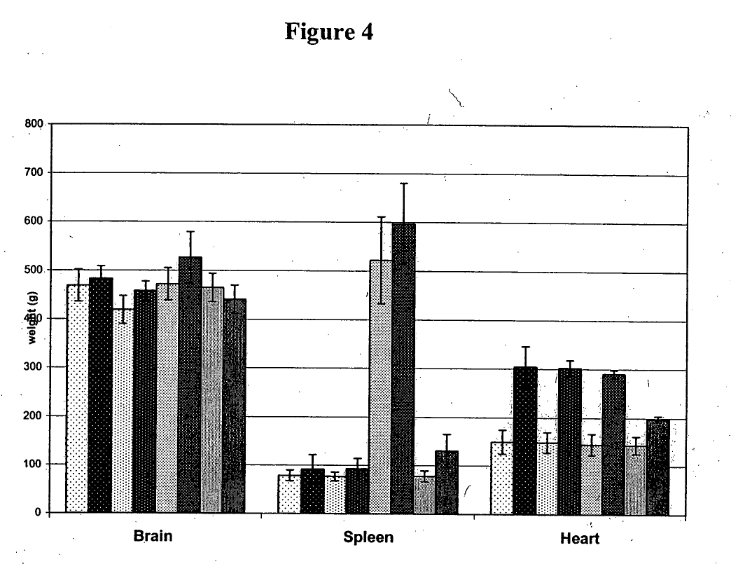

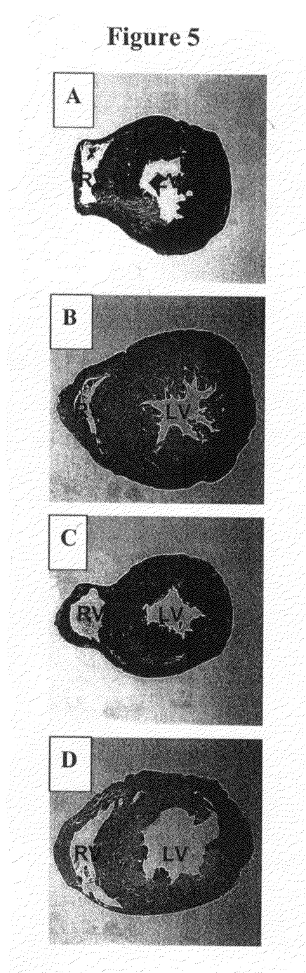

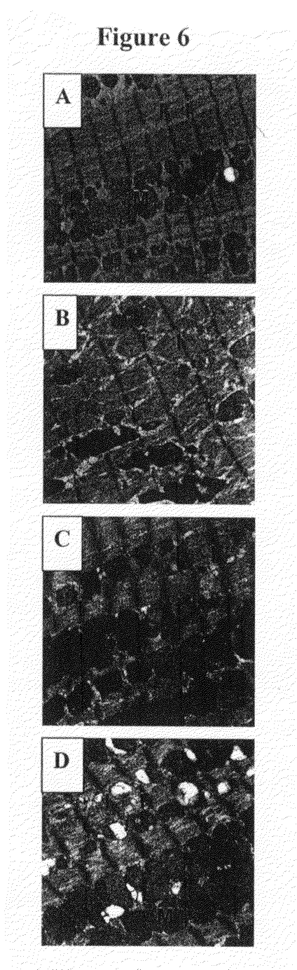

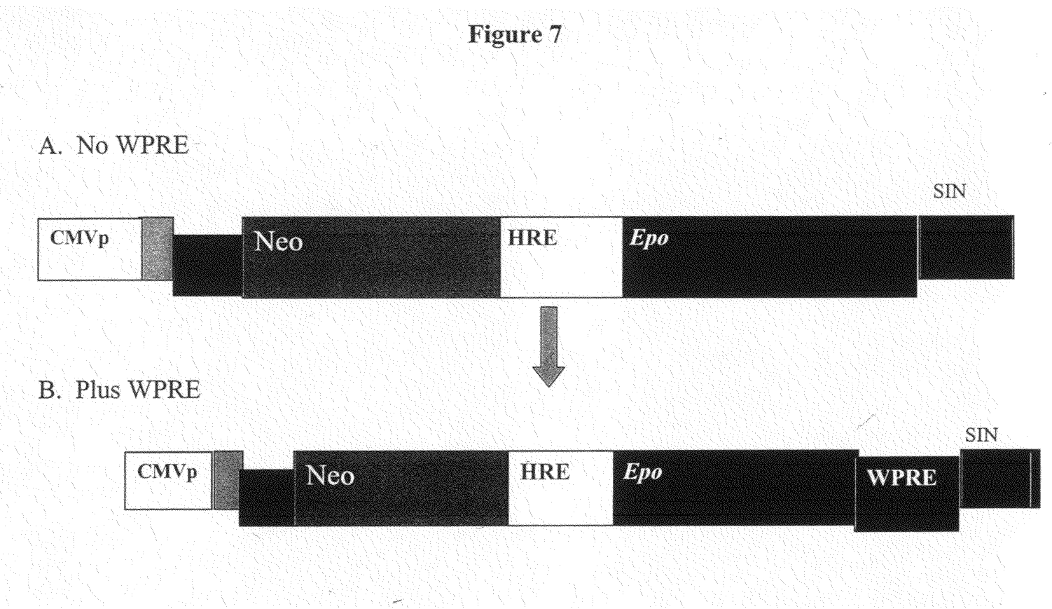

The weights of some of the organs in the untreated and treated EpoTAg^{h} and normal mice (FIG. 4) were compared. There was no difference between any of the groups in the size of the brains. However, marked differences were noted in the spleen. The EpoTAg^{h} mice had spleens that were 70% smaller than the normal mice consistent with the reduction in circulating RBCs. The AAV-CMVEpo treated normal and EpoTAg^{h} mice had massively enlarged spleens (splenomegaly), most likely as a result of vascular congestion due to the increase in RBC load. Splenomegaly has a high incidence (70%) in patients suffering from polycythemia.

There was a doubling of the heart size in the EpoTAg^{h} mice compared to normal consistent with anemia associated hypertrophy. Over-production of Epo from the AAV-CMVEpo vectors caused a further 30% increase in the heart weight of the EpoTAg mice and caused the hearts of the normal mice to increase by 56%. This is presumably due to vascular congestion causing edema in these organs.

Ultrastructure analysis of the hearts confirmed gross hypertrophy in the EpoTAg^{h} mice. Hypertrophy is an increase in the size of a tissue due to increased size of individual cells. It occurs in tissues made up of permanent cells, in which a demand for increased metabolic activity cannot be met through cell multiplication.

In summary, this study describes the surprising and unexpected results obtained by the physiologically-regulated expression of Epo by an HRE. In particular, this study supports that gene therapy by delivery of a recombinant HREEpo vector provides long-term physiologically-regulated expression of Epo for correction of the hematocrit in a genetically anemic environment, without the requirement for any other external intervention or management.

### REFERENCES

### Maxwell et al. (1993) Kidney Int. 44: 1149-1462

### Ebert et al. (1999) Blood 94: 1864-1877

Wenger et al. (1997) Biol. Chem. 378: 609-616

### Binley et al.(1999) Gene Ther. 6: 1721-1727

### Griffiths et al. (2000) Gene Ther. 255-262

### Shibata et al. (2000) Gene Ther. 7: 493-498

### Goodnough et al. (2000) Blood 96: 823-833

Bron et al. (2001) Semin. Oncol. 28: 1-6

### Osada et al.(1999) Kidney International 55: 1234-1240

Dalle et al. (1997) Hematol. Cell Ther. 39: 109-113

### Bohl et al. (1998) Blood 92: 1512-1517

Rudich et al. (May 2000) J. Surg. Res. 90: 102-108

### Zhou et al. (May 1998) Gene Ther. 5: 665-670

Svennson et al. (October 1997) Hum. Gene Ther. 8: 1797-1806

### Beall et al. (March 2000) Gene Ther. 7: 534-539

Payen et al. (March 2001) Exp. Hematol. 29: 295-300

### Tripathy et al. (November 1994) PNAS 91: 11557-11561

Klinman et al. (March 1999) Hum. Gene Ther. 10: 659-665
Maione et al. (April 2000) Hum. Gene Ther. 11: 859-868
Descamps et al. (August 1994) Hum. Gene Ther. 5: 979-985

### Maruyama et al. (March 2001) Gene Ther. 8: 461-468

### Verma (1999) J. Gene Med. 1: 64-66

### Kessler et al. (November 1996) PNAS 93: 14082-14087

### Seppen et al. (August 2001) Blood 98: 594-596

Bohl et al. (1997) Nat. Med. 3: 299-305

### Osborne et al. (August 1995) PNAS 92: 8055-8058

### Villeval et al. (August 1994) Blood 84: 928-933

Serguera et al. (1999) Hum. Gene Ther. 10: 375-383

### Bohl et al. (2000) Blood 95: 2793-2798

### Dalle et al. (1999) Gene Ther. 6: 157-161

Hamamori et al. (1995) J. Clin. Invest. 95: 1808-1813

### Ye et al. (1999) Science 283: 88-91

### Setoguchi et al. (1994) Blood, 94: 2946-2953

### Aebischer et al. (1997) Human Gene Therapy, 8(16): 1840-1841

### Semenza et al. (1992) PNAS 88: 5680-5684

Blanchard et al. (1992) Mol. Cell. Biol. 12: 5373-5385

### Wen et al. (1993) Blood 82: 1507-1516)

Shoemaker et al. (1986) Mol. Cell. Biol. 6: 849-858

### Suliman et al. (1996) Gene 171: 275-280

MacLeod et al. (1998) Am. J. Vet. Res. 59: 1144-1148

### Kessler et al. (1996) PNAS 93: 14082-14087

Krystal (1983) Exp. Hematol. 11: 649-660
Zhang et al. (1999) Hum. Gene Ther. 10: 2527-2537
Boast et al. (1999) Hum. Gene Ther. 10: 2197-2208

### Kaufman et al. (1998) N Engl J Med 339: 578-583

### Anemia Work Group. (1997) Am J Kidney Dis. 30(4 suppl 3): S192-S240

### Asha et al. (1993) Am J Kid Dis. 22(2 suppl 1): 23-31

### Raine et al. (1991) Am J Kidney Dis. 18(4 suppl. 1): S76-S83

### Besarab et al. (1998) N Engl J Med. 339: 584-590

### Watson et al. (1990) Am J Med. 89: 432-435

Having thus described in detail preferred embodiments of the present invention, it is to be understood that the invention defined by the appended claims is not to be limited by particular details set forth in the above description as many apparent variations thereof are possible without departing from the spirit and scope thereof.

We claim:

1. A method for treating anemia in a patient in need thereof, the method comprising administering to the patient a vector comprising a nucleic acid sequence encoding erythropoietin (Epo), in operable linkage with a hypoxia responsive element (HRE) expression control sequence, wherein Epo is expressed and hematocrit levels of the patient are corrected and maintained within normal ranges.

2. The method of claim 1, wherein the vector is a viral vector.

3. The method of claim 2, wherein the viral vector is an adeno-associated viral vector.

4. The method of claim 2, wherein the viral vector is a lentiviral vector.

5. The method of claim 1, wherein the HRE expression control sequence is an Epo HRE expression control sequence.

6. The method of claim 1, wherein the HRE expression control sequence is a PGK-1 HRE expression control sequence.

7. The method of claim 1, wherein the HRE expression control sequence is an LDH-A HRE expression control sequence.

8. The method of claim 1, wherein the HRE expression control sequence is in operable linkage with a promoter.

9. The method of claim 8, wherein the promoter is a viral promoter.

10. The method of claim 9, wherein the viral promoter is the CMV promoter.

11. The method of claim 1, wherein the vector comprises two or more HRE expression control sequences.

12. The method of claim 11, wherein at least one HRE expression control sequence is a PGK-1 HRE expression control sequence.

13. The method of claim 1, wherein the patient is a human.

14. The method of claim 1, wherein the patient is a non-human mammal.

15. The method of claim 14, wherein the patient is a canine, feline, bovine, equine, ovine, porcine or non-human primate.

16. A vector system comprising a nucleic acid sequence encoding erythropoietin (Epo), in operable linkage with two or more HRE expression control sequences, wherein the vector system, when administered to a patient, provides for expression of Epo and hematocrit levels of the patient are corrected and maintained within normal ranges.

17. The vector system of claim 16, wherein the vector system is a viral vector system.

18. The vector system of claim 17, wherein the viral vector system is an adeno-associated viral vector system.

19. The vector system of claim 17, wherein the viral vector system is a lentiviral vector system.

20. The vector system of claim 16, wherein at least one HRE expression control sequence is an Epo HRE expression control sequence.

21. The vector system of claim 16, wherein at least one HRE expression control sequence is a PGK-1 HRE expression control sequence.

22. The vector system of claim 16, wherein at least one HRE expression control sequence is an LDH-A HRE expression control sequence.

23. The vector system of claim 16, wherein the HRE expression control sequences are in operable linkage with a promoter.

24. The vector system of claim 23, wherein the promoter is a viral promoter.

25. The vector system of claim 24, wherein the viral promoter is the CMV promoter.
